(19) United States
(12) Patent Application Publication
(10) Pub. No.: US 2006/0078593 A1
(43) Pub. Date: Apr. 13, 2006

(54) Nutritional compostions comprising a soluble viscous fiber in a solid crisp matrix

(76) Inventors: Deborah C. Strozier, Westerville, OH (US); Chron-Si Lai, Blacklick, OH (US); Sherri Ashton Walker, Sunbury, OH (US); Kay J. Robinson, Westerville, OH (US)

Correspondence Address: ROSS PRODUCTS DIVISION OF ABBOTT LABORATORIES;DEPARTMENT 108140-DS/1, 625 CLEVELAND AVENUE, COLUMBUS, OH, 43215-1724, US

(21) Appl. No.: 11/235,965

(22) Filed: Sep. 27, 2005

Related U.S. Application Data
(60) Provisional application No. 60/613,464, filed on Sep. 27, 2004.

(51) Int. Cl. A61K 36/899; A61K 36/48; A61K 47/00

(52) U.S. Cl. 424/439; 424/757; 424/750

(57) ABSTRACT

Disclosed are nutritional compositions having a solid crisp matrix, or a flour derived from the solid crisp matrix, said matrix comprising (A) from about 10% to about 50% by weight of a soluble viscous fiber, preferably guar; (B) from about 10% to about 99% by weight of a carbohydrate other than and in addition to the soluble viscous fiber; and (C) from about 1% to about 49% by weight of protein, wherein the matrix has a bulk density of less than about 0.4 g/cc. It has been found that soluble viscous fiber sources such as guar gum can be formulated into a solid crisp matrix, which then minimizes several limitations commonly associated with the formulation of such fiber sources into a conventional nutrition bar, e.g. slimy mouthfeel, tooth packing. These compositions are especially effective when formulated with gelled inclusions, including those containing or associated with acidulants, sour flavorants, or both. The nutritional compositions are especially useful as satiety agents, weight reduction agents, and/or for blunting the postprandial glycemic response in diabetics or other individuals in whom such a response would be beneficial.

This application claims the benefit of U.S. Provisional Application No. 60/613,464 filed Sep. 27, 2004.

### FIELD OF THE INVENTION

The present invention relates to nutritional compositions comprising a soluble viscous fiber in a solid crisp matrix. The present invention also relates to methods of using the nutritional compositions as weight loss agents, satiety agents, and to blunt the postprandial response in individuals, especially those afflicted with diabetes.

### BACKGROUND OF THE INVENTION

Many commercially available food products today, including many snack and meal replacement nutrition bars, contain relatively high levels of readily digestible carbohydrates (e.g., high glycemic index carbohydrates). Following consumption, these carbohydrates are rapidly hydrolyzed into glucose and absorbed. Much of the absorbed glucose is then used as either an immediate energy source or is transformed into glycogen in liver or muscle cells, all of which results in a subsequent rise in serum glucose levels as a balance between the amount of glucose absorbed less the amount used immediately for energy.

In certain individuals, however, most notably those afflicted with diabetes, the rise in serum glucose following consumption of such food products can be excessive, the long-term consequences of which may include a variety of neural, vascular, and renal pathologies. And even in individuals who are otherwise not afflicted with diabetes, it is believed that excessive intake of carbohydrates having relatively high glycemic indices may result in excessive weight gain and a negative impact on long-term health.

Consequently, there has been considerable research in recent years directed to the development of food products that will not readily result in hyperglycemia in individuals afflicted with diabetes, and which also can be used as low-glycemic index foods for use in otherwise healthy individuals to promote long-term health benefits. One such research area of particular interest and promise has been the use of various dietary fibers to provide improved blood glucose control, especially in the management of diabetes.

Viscous soluble fibers as a dietary ingredient are well known for their effect in controlling postprandial blood glucose concentrations. These fibers are effective in this respect by forming a viscous digesta following consumption, which then delays stomach emptying, and thus delays or limits glucose absorption in the upper small intestine. It is also believed the soluble fibers like those found in guar gum may also interact with or entangle starch molecules in a manner that retards in vivo amylolysis, and thus results in a further blunting of subsequent serum glucose levels. Although the use of viscous soluble fibers such as guar gum to control blood glucose concentrations is well known, the formulation of these fibers into a palatable and clinically effective dietary product has heretofore been a challenge. Dietary products containing clinically effective concentrations of guar, for example, are often described as unpalatable, especially solid dietary products such as snack or meal replacement bars which, due to the presence of the guar gum, result in a slimy mouth-feel and excessive tooth-packing.

One method of formulating a more palatable yet clinically effective dietary product containing guar gum involves the use of granulated guar gum specifically designed to hydrate slowly after consumption and therefore minimize palatability issues, which are generally associated with the partial hydration of guar gum during chewing. However, although the granulated guar was more palatable than other more conventional guar gum sources, it was not clinically effective in blunting glycemia, presumably because the guar was not sufficiently hydrated after leaving the stomach to be effective.

Although there are currently many different dietary products containing guar gum, including some snack or meal replacement bars, none of the solid formulations contain clinically effective concentrations of a viscous soluble fiber such as guar, while also being acceptably palatable in the targeted population for which the product was designed. Guar and other viscous fibers are most typically used in these formulations at relatively low concentrations as suspending or thickening agents.

It has now been found, however, that a nutritional bar can be formulated with a viscous soluble fiber, which is both palatable and clinically effective in controlling blood glucose levels following a snack or meal. To produce such a product, the nutritional bar must be formulated with a solid crisp matrix, or a soy-fiber flour derived from the solid crisp matrix, such that the solid crisp matrix comprises (1) from about 10% to about 50% by weight of a soluble viscous fiber; (2) from about 10% to about 99% by weight of a carbohydrate; and (3) from about 1% to about 49% by weight of protein, wherein the solid crisp matrix has a bulk density of less than 0.4 g/cm³. It has been found that by formulating the viscous fiber into a solid crisp matrix component of a solid nutritional bar, that the resulting nutritional bar is both palatable and clinically effective in controlling blood glucose levels.

### SUMMARY OF THE INVENTION

The present invention is directed to nutritional compositions having a solid crisp matrix, said matrix comprising (A) from about 10% to about 50% by weight of a soluble viscous fiber; (B) from about 10% to about 89% by weight of an additional carbohydrate; and (C) from about 1% to about 49% by weight of protein, wherein the solid crisp matrix has a bulk density of less than about 0.40 g/cm³. The nutritional compositions are useful as satiety agents, weight reduction agents, and for blunting the postprandial glycemic response in diabetics or other individuals in whom such a response would be beneficial.

The present invention is also directed to a soy-fiber flour derived from the solid crisp matrix described herein. The soy-fiber flour thus comprises a soluble viscous fiber, soy protein, and a second carbohydrate in addition to the soluble viscous fiber, wherein the soy-flour is prepared by forming a solid crisp matrix comprising (i) from about 10% to about 50% by weight of the soluble viscous fiber, (ii) from about 10% to about 89% by weight of a carbohydrate other than and in addition to the soluble viscous fiber, and (iii) from about 1% to about 49% by weight of the soy protein, and then (B) pulverizing or otherwise reducing the solid crisp matrix to form a soy-fiber flour. The present invention is also directed to a solid nutritional composition comprising from about 1% to about 35% by weight of the resulting soy-fiber flour of the present invention.

It has been found that soluble viscous fibers such as guar gum can be formulated into a solid crisp matrix, or a soy-fiber flour derived from the matrix, which then minimizes several limitations commonly associated with the formulation of such soluble viscous fibers in a conventional solid bar matrix. The nutritional compositions of the present invention provide improved mouth feel, e.g., reduced slimy feeling from the viscous fiber, and /or reduced tooth packing as compared to other similar compositions based upon a solid bar matrix.

Although it is well known that solid nutrition bars can be formulated with a solid crisp matrix, it was not previously disclosed that a solid nutritional bar could be formulated with from about 10% to about 50% by weight of a soluble viscous fiber, to improve the slimy mouth feel and reduce the tooth packing problems commonly associated with the use of such fibers in solid nutritional product. And although many different nutritional bars are currently available with a solid crisp matrix and a soluble viscous fiber such as guar, these viscous fibers are formulated either outside the solid crisp matrix or are formulated within the matrix at relatively low concentrations as a processing or formulation aid, not at the relatively high fiber concentrations of from about 10% to about 50% by weight of the solid crisp matrix as described herein, which high concentrations are believed necessary for providing performance benefits such as blood glucose control or as satiety agents or weight reduction dietary products.

It has also been found that the solid crisp matrix can be pulverized or otherwise reduced to form a soy-fiber flour as defined herein, which is also believed to provide improved product performance when formulated into a solid nutritional product.

### BRIEF DESCRIPTION OF THE DRAWINGS

FIG. 1 is a bar chart showing the blood glucose concentrations (AUC) in adult type 2 diabetics following consumption of IVF (Guar crisp embodiment of the present invention), PNE (Pria® Nutritional Energy Bar—NestleUSA, Glendale, Calif.), and CDM (Choice DM® Crispy Snack Bar—Mead Johnson Nutritionals) nutrition bars. Data expressed as mean ±S.E.M.; (**) p<0.001 PNE and CDM>IVF; (*) p<0.001 PNE>CDM.

FIG. 2 is a graph showing the glycemic response in adult type 2 diabetics following consumption of IVF (Guar crisp embodiment of the present invention), PNE (Pria® Nutritional Energy Bar—NestleUSA, Glendale, Calif.), and CDM (Choice DM® Crispy Snack Bar—Mead Johnson Nutritionals) nutrition bars. Data expressed as Mean±S.E.M.; p<0.0001 PNE>IVF and CDM>IVF; PNE>CDM for 0-120 minutes.

FIG. 3. is a graph showing subjective fullness from zero (not at all full) to 100 (extremely full) in adult type 2 diabetics following consumption of IVF Crispy (Guar crisp embodiment of the present invention) or Choice DM® Nutrition Bars (Mead Johnson Nutritionals).

FIG. 4. is a graph showing subjective hunger from zero (not at all hungry) to 100 (extremely hungry) in adult type 2 diabetics following consumption of IVF Crispy (Guar crisp embodiment of the present invention) or Choice DM® Nutrition Bars (Mead Johnson Nutritionals).

FIG. 5. is a graph showing subjective prospective consumption (How much food do you think you could eat right now?) from zero (nothing at all) to 100 (a large amount) in adult type 2 diabetics following consumption of IVF (Guar crisp embodiment of the present invention) or Choice DM® Nutrition Bars (Mead Johnson Nutritionals).

### DETAILED DESCRIPTION OF THE INVENTION

The compositions and corresponding methods of the present invention are directed to nutritional bars or other solid product forms containing a solid crisp matrix as defined herein, or a soy-fiber flour derived from the matrix, which matrix contains as essential ingredients a soluble viscous fiber, an additional carbohydrate, and protein. These and other essential or optional elements or limitations of the compositions and methods of the present invention are described in detail hereinafter.

The term “solid crisp matrix” as use herein, unless otherwise specified, is a term of art within the nutrition formulation art which refers to light, crispy food products having a low bulk density character similar to rice crisps, corn crisps, or similar other well known carbohydrate-containing materials and which has a bulk density of less than about 0.4 g/cm³, preferably less than about 0.35 g/cm³, even more preferably from about 0.10 g/cm³ to about 0.30 g/cm³, and even more preferably from about 0.22 g/cm³ to about 0.28 g/cm³, including from about 0.24 g/cm³ to about 0.27 g/cm³. The term “solid crisp matrix” includes free flowing crisp particulates, bound aggregates of such particulates, and/or solid bar-like matrices, provided that all such particulates, aggregates, or matrices also have the requisite bulk density character as described herein.

The term “lipid” as used herein, unless otherwise specified, means fats, oils, and combinations thereof, excluding the gamma linolenic acid component, which is accounted for separately in the compositions hereof.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

Any reference to singular characteristics or limitations of the present invention shall include the corresponding plural characteristic or limitation, and vice versa, unless otherwise specified or clearly implied to the contrary by the context in which the reference is made.

Any combination of method or process steps as used herein may be performed in any order, unless otherwise specified or clearly implied to the contrary by the context in which the referenced combination is made.

The compositions and methods of the present invention may comprise, consist of, or consist essentially of the essential elements and limitations of the invention described herein, as well as any additional or optional ingredients, components, or limitations described herein or otherwise useful in a nutritional or pharmaceutical application.

The compositions of the present invention may also be substantially free of any optional or selected essential ingredient or feature described herein, provided that the remaining composition still contains all of the required ingredients or features as described herein. In this context, the term “substantially free” means that the selected composition contains less than a functional amount of the optional ingredient, typically less than about 1% by weight, more typically less than about 0.1% by weight, and also including zero percent by weight, of such optional or selected essential ingredient.

The infant formulas and corresponding methods of the present invention can comprise, consist of, or consist essentially of the essential elements and limitations of the invention described herein, as well as any additional or optional ingredients, components, or limitations described herein or otherwise useful in nutritional formula applications.

### Solid Crisp Matrix

The nutritional compositions of the present invention are directed to solid dietary product forms, preferably snack or meal replacement bars, comprising the solid crisp matrix as defined herein. Any solid dietary product form, known or otherwise, is suitable for use herein provided that it can be modified to contain one or more of the solid crisp matrices as defined herein.

The solid crisp matrix of the nutritional composition of the present invention must comprise, however, 1) from about 10% to about 50% by weight of a soluble viscous fiber, 2) from about 10% to about 89% by weight of a carbohydrate other than and in addition to the soluble viscous fiber, and 3) from about 1% to about 49% by weight of protein. Each of these essential components of the solid crisp matrix is described hereinafter in greater detail.

The nutritional composition of the present invention may comprise a combination of the solid crisp matrix along with one or more other solid crisp materials, wherein the other solid crisp material does not contain a soluble viscous fiber or does not otherwise contain more than about 9% by weight of a soluble viscous fiber. The other solid crisp material may represent within the nutritional composition from zero percent to about 99%, including from about 5% to about 90%, also including from about 20% to about 80%, and also including from about 30% to about 50%, by weight of the combination of the solid crisp matrix and the solid crisp material in the composition. Such other solid crisp material includes any conventional or otherwise known grain-based crisp, preferably having a bulk density within the ranges described herein for the essential solid crisp matrix component of the nutritional composition. Non-limiting examples of such other solid crisp materials include soy crisps, rice crisps, corn crisps, tapioca starch in crisp form, various multi-grain crisps, and combinations thereof.

The solid crisp matrix component of the nutritional composition of the present invention, alone or in combination with an additional crisp material as described above, preferably represents from about 5% to 100% by weight of the nutritional composition, including from about 50% to about 98%, and also including from about 75% to about 95%, and also including from about 80% to about 90%, by weight of the nutritional composition. The nutritional composition may also comprise from zero to 95% by weight of optional materials such nuts or seeds, fruit or other flavored materials, processing aids (e.g., binders), antioxidants, vitamins and minerals, and so forth.

The solid crisp matrix as formulated into the nutritional composition may be an aggregate of low bulk density materials or food particles bound together, or a continuous or substantially continuous low bulk density matrix, wherein the solid crisp matrix has a bulk density of less than about 0.4 g/cm³, preferably less than about 0.35 g/cm³, even more preferably from about 0.10 g/cm³ to about 0.30 g/cm³, and even more preferably from about 0.22 g/cm³ to about 0.28 g/cm³, including from about 0.24 g/cm³ to about 0.27 g/cm³.

The bulk density of the solid crisp matrix can be measured or otherwise determined by most any conventional method, wherein the bulk density is the mass or weight (gm) per unit volume (cm³) of the matrix, whether the matrix is a solid porous bar or a formed or free flowing aggregate of smaller solid crisp matrices, with air or gaseous voids dispersed throughout and between the matrices.

The solid crisp matrix may be prepared by any method suitable for making a low bulk density material as described herein. Such methods most typically include the mixing together of the essential and at least some optional materials for forming the crisp (e.g., water, soy protein isolate, guar gum, rice flour, tricalcium phosphate), heating the mixture in a cooking extruder or the cooking section of a cooking expanding extruder, and then extruding the mixture under appropriate pressure and heat to form an expanded, low bulk density, material upon exiting the extruder, e.g., low bulk density food particles, which can then be combined with any other necessary or optional ingredients such as a binding agent, and then formed and cut into the desired bar size and shape. Other suitable methods of making such low bulk density food particles or materials are described, for example, in U.S. Pat. No. 6,676,982 (Mody), which description is incorporated herein by reference.

The nutritional compositions of the present invention may be prepared in most any dietary product forms of any size or configuration, e.g., rounded or cylindrical, circular or wafer-like, rectangular or in a conventional bar form, or random or other defined shapes. These product forms also include small bite size solids, including those that are packaged as a plurality of bites within a single container or package. The solid crisp matrix may also be packaged as free flowing food particles, e.g., breakfast cereal, in an appropriate box or other package.

The nutritional compositions of the present invention may contain one or more layers of the solid crisp matrix, or may otherwise contain one or more discrete regions of the solid crisp matrix in a random, arranged, or patterned configuration. The nutritional composition, as well as the solid crisp matrix therein, may be partially or completely coated with any suitable coating material, some common examples of which include yogurt, chocolate, or other confectionary or otherwise flavored material.

### Soluble Viscous Fiber

The solid crisp matrix of the nutritional compositions of the present invention must comprise from about 10% to about 50%, preferably from about 15% to about 40%, including from about 19% to about 46%, and also including from about 21% to about 32%, of a soluble viscous fiber by weight of the solid crisp matrix. The soluble viscous fiber is preferably guar.

The term “soluble viscous fiber” as used herein, unless otherwise specified, may include any fiber or fiber containing material that is both viscous and soluble as defined herein. A soluble fiber for purposes of the present invention is defined by the American Association of Cereal Chemists (AACC) Method 32-07, wherein a soluble fiber or fiber source is one in which at least 60% of the dietary fiber is soluble dietary fiber as determined by AACC Method 32-07.

The term “viscous fiber” as used herein, unless otherwise specified, refers to a soluble fiber that when formulated into a solid crisp matrix may provide the matrix with an in vivo viscosity greater than about 300 centipoise (cps), including at least about 1,000 cps, also including from about 1,000 cps to about 10,000 cps, and also including from about 3000 cps to about 10,000 cps.

The in vivo viscosity for purposes of characterizing soluble viscous fibers is measured by the following method:
prepare an aqueous mixture containing 3% by weight of the solid crisp matrix (10.4 g solid crisp matrix and 349.34 g water),
blend the just-formed mixture for 1 minute,
raise the blend temperature to about 37° C. by placement in a 37° C. water bath,
add 300 microliters of sigma alpha amylase to the warmed and blended mixture,
allow the mixture to set for one hour, and then rapidly agitate it over about 0.5 minutes to more fully disperse the incubating mixture,
allow the mixture then to set for a second hour, and then immediately transfer approximately 250 cm³ of the mixture to a 250 cm³ beaker, and then measure the viscosity (e.g. of the transferred mixture using a Brookfield viscometer, #62 spindle, at 3 rpm.

Soluble viscous fibers for use in the solid crisp matrix include any fiber or fiber system satisfying the above described criteria for fiber in vivo viscosity and fiber solubility. The soluble viscous fibers may also be defined in the alternative as being a fiber source comprising one or more of guar gum, gum arabic, sodium carboxymethyl cellulose, locust bean gum, tapioca starch, alginates, tapioca dextrins, citrus pectin, low and high methoxy pectin, carrageenan, barley glucans, carrageenan, psyllium, oat β-glucan, and combinations thereof. Guar gum is preferred.

As the preferred soluble viscous fiber herein, guar gum (galactomannan polymer) is a complex carbohydrate derived from the seed of specially grown bean plants. This carbohydrate is a long chain linear molecule with a molecular weight of approximately 1 million. The long polymer chains attract and weakly capture water; as well as physically tangle with one another in solution thus producing viscosity when mixed with water.

Non-limiting examples of suitable sources of the soluble viscous fibers, including sources of guar gum, are available from Tic Gums, 4609 Richlynn Drive, Belcamp, Md., U.S.A 21017 (Guar 8/24, fine mesh, very high viscosity product).

The soluble viscous fiber may also include two or more soluble viscous fibers, including the dual fiber systems described in U.S. Patent Application 20030125301A1 (Wolf et al.), which description is incorporated by reference herein.

It has been found that the viscous soluble fiber must be formulated into the solid crisp matrix to provide the palatability benefits (e.g., reduced slimy mouth feel, reduced tooth packing) described herein. And although minor amounts of the fiber may be found elsewhere in the nutritional composition, the nutrition composition outside the solid crisp matrix may be substantially free of such fibers, including guar. In this context, the term “substantially free means that the composition may contain less than about 3%, including less than about 2%, and also including less than about 0.1%, and also including zero percent, of such fiber within the nutritional composition but outside the solid crisp matrix component, all by weight of the nutritional composition.

### Acidulant and Sour Flavorant

The nutritional compositions of the present invention preferably further comprise an acidulant, a sour flavorant, or both. Any material that provides a sour and/or acidic flavor that is known or otherwise suitable for use in a solid nutritional product may be used in the compositions of the present invention, provided that such materials are safe and effective for oral administration and are compatible with the essential and other ingredients in the selected product form.

It has been found that the acidulant and sour flavorants, especially when used in combination, and even more so when used in combination with pectin pieces as described hereinafter, improves overall mouthfeel and reduces the extent or frequency of tooth packing while chewing and consuming the nutritional compositions. Without being limited by theory, it is believed that the selected acidulants and/or sour flavorants stimulate more salivation than many other flavors (or no flavor at all) and that the increased salivation then acts as a lubricant during chewing to further reduce adhesion of the guar-containing composition onto the surfaces of the teeth, especially on the crevaced chewing surfaces where undesirable tooth packing most often occurs.

Acidulants suitable for use in the compositions of the present invention include any organic or inorganic edible acid in undissociated form or, alternatively, as their respective salts, for example, potassium or sodium hydrogen phosphate, potassium or sodium dihydrogen phosphate salts, and so forth. Non-limiting examples of suitable acidulants include citric acid, phosphoric acid, malic acid, fumaric acid, adipic acid, gluconic acid, tartaric acid, ascorbic acid, acetic acid, phosphoric acid, and combinations thereof. Acidulant concentrations in the composition most typically exceed about 0.01% by weight of the composition, more typically from about 0.05% to about 3%, including from about 0.1% to about 1.0%, by weight of the composition.

Sour flavorants suitable for use in the compositions of the present invention include any natural or artificial favor or combination of flavors, which provides the composition with sufficient sour notes to be detected during consumption. Non-limiting examples of such sour flavorants include pieces or extracts of, or natural or artificial flavors based upon, natural materials such as strawberry, apple, blueberry, raspberry, blackberry, cherry, orange, lime, lemon, grapefruit, tangerine, bergamot, calamondin, chironja, citron, clementine, dancy, kumquat, limequat, mandarin orange, mandarin lime, minneola, orangelo, orangequat, pummelo, rangpur, satsuma, shaddock, shekwasha, sweety, tangelo, tangor, ugli, or other plant materials containing one or more of the organic acidulants as described herein. Especially useful are dried strawberry pieces (flakes) and/or apple pieces.

Flavorant concentrations can vary considerably depending upon the flavorant, other ingredients in the composition, the desired overall flavor profile of the composition, whether the flavor comprises whole fruit or only an extract therefrom, and so forth. Such concentrations, however, most typically and collectively range from at least about 0.01%, more typically from about 0.05% to about 10%, also including from about 0.1% to about 5%, and also including from about 0.5% to about 4%, by weight of the nutritional composition.

### Gelled Inclusion

The nutritional compositions of the present invention may further comprise one or more gelled inclusions, wherein the inclusions comprise water and not more than about 9%, including from about 0.5% to about 7%, and also including from about 1.5% to about 5%, of a primary gellant by weight of the inclusions, and preferably an acidulant, sour flavorant, or both.

The term “gelled inclusion” as used herein refers to separate gelled structures that are prepared prior to final formulation, and then added to the nutritional composition as a component of the solid crisp matrix, or as a component separate from the solid crisp matrix. The gelled inclusions can take the form of many gelled particulates or pieces collectively dispersed throughout the solid crisp matrix or other areas of the nutritional composition, or it can take the form of one or a few larger discrete regions or layers which represent a large, continuous gelled inclusion(s), e.g., a gelled layer(s) on top of or within a nutritional bar embodiment.

Once added to and formulated into the composition, especially when dispersed as individual particulates throughout the solid crisp matrix or other component of the composition, the gelled inclusions may lose much if not all of their gelled structure, but still form discreet areas or regions within the nutritional composition defined by the presence of the selected gellant and any other ingredients specifically formulated into the gelled particulates prior to formulating into the nutritional composition. Such other ingredients preferably include acidulants, sour-flavorants, or combinations thereof, but may also comprise any of a variety of other optional ingredients such as other flavorants, flavor enhancers, artificial or natural sweeteners, sugar alcohols, etc.

The gelled inclusions preferably represent at least about 1.0% by weight of the nutritional composition, more preferably from about 1.2% to about 15%, including from about 2% to about 11%, and also including from about 5% to about 9%, by weight of the composition.

The gellant for use in the gelled inclusions may be any gellant material safe and effective for use in a nutritional composition, and which is otherwise compatible with the other selected ingredients as formulated within the nutritional composition. Preferred gellants include the viscous soluble fibers as described herein, to the extent such fibers can form an aqueous gel at the requisite gellant concentration described above. Among the viscous soluble fibers described herein, pectin is most preferred.

The gelled inclusions are preferably formulated into the nutritional composition in combination with the optional but preferred acidulant, sour flavorant, or both, all as described hereinbefore. The gelled inclusions as particulates are preferably prepared so that the various particulates contain an acidulant and/or sour flavorant, although it is understood that all or some of such acidulants and sour flavorants can also be formulated into the nutritional compositions separate from the gelled inclusion, although the formulation with the gelled inclusion is preferred. When used in combination with a sour flavorant and/or acidulant, the preferred pectin gellant is also preferably a highly methoxylated pectin, most typically those having a degree of esterification of less than about 65%, including less than about 50%.

It has been found that the gelled inclusions such as those containing pectin pieces or other similar particulates, especially when used in combination with an acidulant and sour flavorant, provides for even better performance in reduced tooth packing during consumption, and improved mouthfeel. For purposes of defining the compositions of the present invention, therefore, the concentration of the viscous soluble fiber in the crisp solid matrix is considered separate from the concentration of the viscous soluble fiber or gellant concentration provided by the gelled inclusion.

Non-limiting examples of some gelled inclusions suitable for use in the compositions of the present invention, including those containing sour/acid flavored pectin pieces suitable for use herein, include Fantasy® Strawberry NSA Fruit Pieces, Artificial; Natural and Artificial Chocolate Peanut Pieces, NSA; Natural and Artificial Butter-Pecan Pectin Pieces, NSA; Natural and Artificial Espresso Pectin Pieces, NSA; Natural Apple Cinnamon Pectin Pieces, NSA; all of which are available from Sensient, Indianapolis, Ind., U.S.A. Other non-limiting examples of suitable flavored pectin pieces include Realfruitchips, Raspberry No Sugars added- Low Net Carbs, available from Brookside Foods, Ltd., Abbotsford, British Columbia, Canada.

### Protein

The solid crisp matrix of the nutritional compositions of the present invention must also comprise a protein or protein source, which represents from about 1% to about 49%, preferably from about 5% to about 43%, including from about 20% to about 40%, and also including from about 30% to about 39%, by weight of the solid crisp matrix. The protein or protein source may be any known or otherwise suitable source that is safe and effective for oral administration and is compatible with the essential and other ingredients in the selected product form.

Protein or protein sources suitable for use in the nutritional compositions include hydrolyzed, partially hydrolyzed or non-hydrolyzed proteins or protein sources, and can be derived from any known or otherwise suitable source such as milk (e.g., casein, whey), animal (e.g., meat, fish, egg albumen), cereal (e.g., rice, corn), vegetable (e.g., soy, pea, potato), or combinations thereof. The proteins for use herein can also include, or may be entirely or partially replaced by, free amino acids known for use in nutritional products, non-limiting examples of which include tryptophan, glutamine, tyrosine, L-methionine, cysteine, taurine, L-arginine, camitine, and combinations thereof.

The nutritional compositions of the present invention preferably comprise a soy protein component, sources of which include, but are not limited to, soy flakes, soy protein isolates, soy protein concentrate, hydrolyzed soy protein, soy flour, soy protein fiber, or any other protein or protein source derived from soy. Commercial sources of soy protein are well known in the nutrition art, some non-limiting examples of which include soy protein isolates distributed by The Solae Company under the trade designation “Soy Protein Isolate EXP-H0118,” “EXP-E-0101, and “Supro Plus 675.”

### Carbohydrate

The solid crisp matrix of the nutritional compositions of the present invention must also comprise a carbohydrate source other than an in addition to the soluble viscous fiber component described herein. The additional carbohydrate source within the matrix represents from about 10% to about 89%, including from about 10% to about 70%, also including from about 20% to about 60%, and also including from about 30% to about 50%, by weight of the solid crisp matrix. The carbohydrate source may be any known or otherwise suitable source that is safe and effective for oral administration and is compatible with the essential and other ingredients in the selected product form.

Suitable carbohydrates or carbohydrate sources for use in the solid crisp matrix may be simple, complex, or variations or combinations. Non-limiting examples of suitable carbohydrates include hydrolyzed or modified starch or cornstarch, maltodextrin, glucose polymers, oligosaccharides (e.g., fructooligosacchardes, glucooligosaccharides), sucrose, corn syrup, corn syrup solids, rice-derived carbohydrate, glucose, fructose, lactose, high fructose corn syrup, honey, sugar alcohols (e.g., maltitol, erythritol, sorbitol), and combinations thereof.

Other suitable carbohydrates include any dietary fiber or fiber source other than and in addition to the soluble viscous fiber, non-limiting examples of which include insoluble dietary fiber sources such as oat hull fiber, pea hull fiber, soy hull fiber, soy cotyledon fiber, sugar beet fiber, cellulose, corn bran, and combinations thereof.

The carbohydrate for use in the solid crisp matrix may therefore include soluble and/or insoluble fiber, or other complex carbohydrate, preferably having a DE (dextrose equivalent) value of less than about 40, including less than 20, and also including from 1 to 10.

### Lipid

The solid crisp matrix of the nutritional compositions of the present invention may optionally comprise a lipid or lipid source, which may represent from zero to 70%, including from about 0.1% to about 65%, and also including from about 0.5% to about 50%, and also including from about 2% to about 45%, and also including from about 5% to about 30%, by weight of the lipid or lipid source. The lipid or lipid source may be any known or otherwise suitable source that is safe and effective for oral administration and is compatible with the essential and other ingredients in the selected product form.

Optional lipids or lipid sources suitable for use in the solid crisp matrix include coconut oil, fractionated coconut oil, soy oil, corn oil, olive oil, safflower oil, high oleic safflower oil, MCT oil (medium chain triglycerides), sunflower oil, high oleic sunflower oil, palm and palm kernel oils, palm olein, canola oil, marine oils, faxseed oil, borage oil, cottonseed oils, evening primrose oil blackcurrant seed oil, transgenic oil sources, fungal oils, marine oils (e.g., tuna, sardine) and so forth. Other suitable lipids include both essential and non-essential fatty acids, including omega-3 fatty acids, omega-6 fatty acids, and combinations there.

### Macronutrient Profile

The total amount or concentration of lipid, carbohydrate, and protein, in the nutritional compositions of the present invention can vary considerably depending upon the selected formulation and dietary or medical needs of the intended user. In this context, the total amount or concentration refers to all lipid, carbohydrate, and protein sources in the nutritional composition, including those within the solid crisp matrix and those outside or not part of the solid crisp matrix. Such total amounts or concentrations are most typically and preferably formulated within any of the embodied ranges described in the following table.

Macronutrients*

| | Percentage of | | | Wt/wt percent of | | |
|---|---|---|---|---|---|---|
| | total calories | | | Nutritional Composition | | |
| Nutrients | A | B | C | A | B | C |
| Carbohydrate | 5-95 | 10-70 | 40-50 | 5-95 | 1-50 | 10-30 |
| Lipid | 0-100 | 20-65 | 35-55 | 0-90 | 1-30 | 3-15 |
| Protein | 5-95 | 5-40 | 15-25 | 5-95 | 1-20 | 2-10 |

*Each numerical value is preceded by the term “about”

### Optional Ingredients

The compositions of the present invention may further comprise other optional components that may modify the physical, chemical, aesthetic or processing characteristics of the products or serve as pharmaceutical or additional nutritional components when used in the targeted population. Many such optional ingredients are known or otherwise suitable for use in medical food or other nutritional products or pharmaceutical dosage forms and may also be used in the compositions herein, provided that such optional ingredients are safe and effective for oral administration and are compatible with the essential and other ingredients in the selected product form.

Non-limiting examples of such optional ingredients include preservatives, anti-oxidants, emulsifying agents, buffers, pharmaceutical actives, additional nutrients as described herein, sweeteners including artificial sweeteners (e.g., saccharine, aspartame, acesulfame K, sucralose) colorants, flavorants in addition to those described herein, thickening agents and stabilizers, emulsifying agents, lubricants, and so forth.

The nutritional compositions of the present invention may further comprise in addition to and separate from the materials in the solid crisp matrix, various combinations of the different lipid, carbohydrate, and protein materials described herein, as well as additional vitamins, minerals, or other nutrients.

Non-limiting examples of suitable minerals for use herein include phosphorus, sodium, chloride, magnesium, manganese, iron, copper, zinc, iodine, calcium, potassium, chromium, molybdenum, selenium, and combinations thereof.

Non-limiting examples of suitable vitamins for use herein include carotenoids (e.g., beta-carotene, zeaxanthin, lutein, lycopene), biotin, choline, inositol, folic acid, pantothenic acid, choline, vitamin A, thiamine (vitamin B₁), riboflavin (vitamin B₂), niacin (vitamin B₃), pyridoxine (vitamin B₆), cyanocobalamine (vitamin B₁₂), ascorbic acid (vitamin C), vitamin D, vitamin E, vitamin K, and various salts, esters or other derivatives thereof, and combinations thereof.

Food Particulates

The compositions of the present invention include embodiments formulated as free flowing crisp particulates, which may be a final product form or an intermediate material from which other products may be formulated, such as various solid bar embodiments of the present invention. These free flowing crisp particulates comprise any of the solid crisp formulations of the present invention, which may further comprise any of the optional ingredients also described herein.

The free flowing crisp particulates may be coated using any material suitable for application to such particulates while also maintaining the free flowing character of such particulates. Such coating materials may be film-forming or non-film-forming materials, most of which are either biopolymers (proteins and polysaccharides) or lipids. Non limiting examples of such coating materials include gluten (e.g., wheat gluten), milk proteins, soy proteins, gelatin, starch (e.g., hydroxypropylated starch), pectinates, cellulose-ethers, hydrophobic fats or waxes, and combinations thereof.

The free flowing crisp particulates may be used as food additives sprinkled onto or mixed within various foods, consumed alone or in combination with other food or beverages as a snack or satiety agent, especially prior to meals. The free flowing crisps may be used as a formulation intermediate in the preparation of other food products such as snack or meal replacement bars or other consumer food products. As a food additive for sprinkling onto or mixing with foods, the free flowing crisp particulates may be formulated with conventional seasoning or other flavors to provide a seasoned or other flavored food additive in particulate form.

When formulated for use as a snack or satiety agent, for use prior to or with a meal, the free flowing crisp particulates include those embodiments comprising in a single dose of up to about 100 kcals, including from 25 to 100 kcals, also including from 40 to 75 kcal, from at least about 6 grams per dose, including from about 7 to about 16 grams, and also including from about 8 to about 12 grams per dose.

Optional ingredients especially useful in these free flowing particulates include sugar alcohols (e.g., maltitol, erythritol, sorbitol, xylitol, mannitol, glycerol, isolmalt, lactitol) or other low glycemic index ingredients, seasoning, phytosterols, glycomacropeptide, and so forth, all of which may be formulated within or on (i.e., coating) the crisp particulates.

For coated particulates, the coating may represent up to 25% by weight of the finished product, including from about 5 to about 20%, and including from about 8 to about 14%, by weight of the finished product.

### Methods of Use

The methods of the present invention are all directed to the nutritional compositions of the present invention. These methods include the following:
1. The oral administration of the nutritional compositions to individuals to provide a balanced or complete source of nutrition.
2. The oral administration of the nutritional compositions to diabetics or other individuals to provide a blunting of the glycemic response following administration of a snack or meal.
3. The oral administration of the nutritional compositions to diabetics or other individuals to help reduce appetite.
4. The oral administration of the nutritional compositions to diabetics or other individuals to help reduce total body weight or total fat content of the individual.

The methods of the present invention preferably comprise the daily administration of at least one serving of the nutritional composition, in single or divided doses, in the diabetic or other individual to whom the benefits of such administration would be useful. In this context, a serving is defined as the total daily amount of the nutritional composition to be administered to the individual, which is most typically in the form of from about 1 to about 6 bars per day, for a total daily caloric intake from the composition of at least about 50 kcal/day, more typically from about 50 kcal/day to about 3,000 kcal/day, and even more typically from about 120 kcal/day to about 600 kcal/day.

The nutritional composition of the present invention for use in the various methods is preferably a bar formulation comprising a combination of lipid, protein, carbohydrate, vitamins, and minerals, and more preferably comprises from about 99 kcal to about 350 kcal, more preferably from about 120 kcal to about 280 kcal, per individual bar.

### Manufacture

The nutritional compositions of the present invention may be prepared by any known or otherwise effective manufacturing technique for preparing the selected solid product form containing a solid crisp matrix as defined herein. Many such techniques are known for any given product form such as coated or uncoated, layered or unlayered, nutritional bars, and can easily be applied by one of ordinary skill in the art to the nutritional compositions described herein.

The solid crisp matrix for use in the composition can be prepared by any known or otherwise suitable method for preparing a grain-based crisp. Such crisps include toasted puffed grains prepared by mixing rice or other grain kemals (whole or pieces) with a flavor solution (e.g., sugar, syrups, salt, water) and other optional ingredients (e.g., vitamins, minerals) and then cooking, drying, cooling, tampering, and toasting the crisp material.

The solid crisp matrix may also be prepared by any known or otherwise suitable extrusion process such as high temperature short time (HTST) extrusion as a continuous cooking process. The crisp ingredients are combined (e.g., rice flour, guar gum, tricalcium phosphate, maltodextrin, soy protein isolate, water), and the combination metered into a feed line with additional moisture (steam) to an extrusion barrel and conveyed forward by a screw or a series of screws. Within the screw, there is a groove that becomes progressively shallower towards the exiting end of the barrel. The mechanical energy, imparted to the feed, is transformed into heat to cook the feed. To further facilitate this cooking, the barrel can be heated. This combination of moisture and heat transforms the feed powder into a dough. When the temperature of the dough exceeds 100° C., the water becomes super-heated. And as the dough exits the die, the super-heated water explodes and causes expansion of the dough. This expanded dough can be cut into small pieces and dried or toasted which results in a solid crisp matrix for use in the nutritional compositions of the present invention. The process is controlled by conventional means to deliver the requisite bulk density. Some suitable methods of making such low bulk density food particles or materials in forming a solid crisp matrix are described, for example, in U.S. Pat. No. 6,676,982 (Mody), which description was previously incorporated herein by reference.

The solid crisp matrix, regardless of the method used in preparing it, is most typically in the form of individual, free flowing, crispy particles, which can then be combined to form a continuous or discontinuous solid matrix within the nutritional composition, all of which may be prepared by conventional bar manufacturing methods using such food particle crisps as an ingredient.

In general, the nutritional bars and other solid compositions of the present invention are most typically manufactured by conventional methods commonly used for non-baked nutrition bars. For example, the various syrups, oils, binders, and sugars (e.g., fructose) are combined together with agitation and heated to about 140° F., and then fed into a mixer and combined with the solid crisp matrices and other ingredients, the result of which is then slabbed (e.g., 0.5-1.0 inch sheets), cut into the desired shapes, optionally coated, cooled, and then packaged.

The compositions of the present invention may, of course, be manufactured by other known or otherwise suitable techniques not specifically described herein without departing from the spirit and scope of the present invention. The present embodiments are, therefore, to be considered in all respects as illustrative and not restrictive and that all changes and equivalents also come within the description of the present invention. The following non-limiting examples will further illustrate the compositions and methods of the present invention.

### EXAMPLES

The following examples illustrate specific embodiments of the nutritional compositions and methods of the present invention, including some suitable techniques to prepare the compositions. The examples are given solely for the purpose of illustration and are not to be construed as limitations of the present invention, as many variations thereof are possible without departing from the spirit and scope of the invention.

Each of the exemplified compositions provides improved sensory benefits, and is used in accordance with the methods of the present invention, including a method of controlling blood glucose concentrations in a diabetic or other individual following a snack or meal, said method comprising the oral administration of any one of the exemplified nutrition bars or other compositions. The compositions are also used to reduce appetite in a diabetic or other individual by the oral administration of any one of the exemplified nutrition bars or other compositions.

Unless otherwise specified, all exemplified ingredient amounts are listed as wt/wt percentages based upon the final weight of the targeted composition, unless otherwise specified.

### Examples 1 and 2

Examples 1 and 2 illustrate guar crisp embodiments of the present invention, each of which is used in accordance with the methods of the present invention. These guar crisps are also used to formulate the nutritional bars described in Examples 3-12.

Examples 1 and 2: Guar Crisps

| Ingredient | Example 1 | Example 2 |
|---|---|---|
| Corn flour | 34.750% | — |
| Rice flour | — | 25% |
| Guar gum, (8/24 TIC) | 30.000% | 30% |
| Tricalcium phosphate | 0.100% | 0.005% |
| Monoglycerides | 0.150% | — |
| Maltodextrin DE 10 | 5.000% | — |
| Soy protein isolate (˜80% protein) | 30.000% | 44.995% |
| Total | 100.000% | 100.000% |

The ingredients for each formulation are mixed together, and then metered into a feed line with additional moisture (steam) to an extrusion barrel and conveyed forward by an extrusion screw. The mechanical energy, imparted to the feed, is transformed into heat to cook or help cook the feed. The extrusion barrel is also heated as needed during the process to facilitate the cooking or heating process. This combination of moisture and heat transforms the feed into dough, and when the temperature of the dough exceeds about 100° C., the water therein becomes super-heated. And as the heated dough under extrusion pressure exits the die, the super-heated water expands and causes a resulting expansion of the surrounding dough. This expanded dough exiting the extrusion barrel is then cut or otherwise reduced into small pieces, and then toasted to form solid guar crisps. The extrusion and baking process is controlled by conventional means to provide a bulk density of from 0.22-0.28 g/cm³. The resulting guar crisps are in the form of individual, free flowing, crispy, low-bulk density food particles.

### Examples 3-6

Examples 3-6 illustrate nutritional bar embodiments of the present invention. Also included are corresponding methods of using the compositions in accordance with the methods of the present invention.

Examples 3-6: Nutrition Bars

| Ingredient | Example 3 | Example 4 | Example 5 | Example 6 |
|---|---|---|---|---|
| Rice Syrup 42 DE, High Maltose | 3.8151% | 3.8038% | 3.0958% | 3.0958% |
| Brown Rice Syrup 42 DE, High Maltose | 1.5373% | 1.5373% | 1.6372% | 1.6372% |
| Energy Smart ® Syrup (mixed fruit juice | 14.1741% | 14.1740% | 15.2486% | 15.2486% |
| concentrates and natural grain dextrins) | | | | |
| Glycerin | 1.1376% | 1.1376% | 1.2113% | 1.2113% |
| Sugar-Free Marshmallow Binder | 13.9550% | 13.8897% | 14.3233% | 14.3233% |
| High Oleic Safflower Oil | 1.5373% | 1.5373% | 1.9643% | 1.9643% |
| Fructose | 0.0976% | 0.0977% | 0.2023% | 0.2023% |
| Cinnamon and Graham Flavor | 0.9306% | 0.9305% | — | — |
| Strawberry Flakes | — | — | 2.0299% | 2.0299% |
| Strawberry Flavor | — | — | 0.4204% | 0.4204% |
| Natural Buttery Flavor | — | — | 0.0766% | 0.0766% |
| Maltodextrin (Fibersol 2) DE8-12 | 2.6321% | 2.7080% | 1.6646% | 1.6646% |
| Vitamin-mineral premix | 0.5188% | 0.5188% | 0.4335% | 0.4335% |
| Ascorbic acid (70% active) | 0.0463% | 0.0463% | 0.4000% | 0.4000% |
| Fructo Oligosaccharide (FOS) | 0.9222% | 0.9222% | 0.9821% | 0.9821% |
| Cellulose Gel-Novagel BK-2132 | 0.6151% | 0.6151% | 0.6550% | 0.6550% |
| Dried Apples (unsulfured evaporated), dcd ¼″ | — | — | 2.0299% | 2.0299% |
| Honey Graham Crumbs | 2.9507% | 2.9514% | — | — |
| Chocolate Chips Sugar Free ¹ | 5.8428% | 6.8713% | — | — |
| Soy Protein Isolate Crisps | 5.3750% | 16.1805% | 6.7103% | 18.5442% |
| Guar Crisps (Example 2) | 23.6678% | 11.8339% | 23.6678% | 11.8340% |
| Strawberry Pectin Pieces | — | — | 2.9078% | 2.9078% |
| Yogurt Coating (no sugar added) | — | — | 20.3393% | 20.3393% |
| Chocolate Coating (no sugar added) | 20.2448% | 20.2448% | — | — |
| Total | 100.000% | 100.000% | 100.000% | 100.000% |

¹ Chocolate chips sprinkled on top of the formed bar after the composition is slabbed

The nutritional bar embodiments of Examples 3-6 are prepared by similar and conventional methods for making a nutrition bar with a crisp matrix base. The crisps used in each exemplified formula are prepared by conventional HTST (high temperature short time) extrusion to a bulk density of from about 0.23-0.28 g/cm³.

The Example 6 formula, for example, is prepared by first combining the high maltose rice syrup, high maltose brown rice syrup, EngergySmart® syrup, glycerin, marshmallow binder, and high oleic safflower in a vessel. The combination is mixed together and heated to 140° F. Strawberry flakes, strawberry flavor, and butter flavor are then added to the heated mixture. The resulting combination is mixed with agitation for about 1 minute, and thereafter a pre-blend is added which contains the maltodextrin fiber (Fibersol-2), oligofructose powder (FOS), cellulose gel, vitamin-mineral premix, ascorbic acid, and crystalline fructose. The resulting mixture is mixed with agitation for about 2 minutes, after which the dried apples, soy fiber crisps, and soy protein crisps are added to the forming mass and mixed with agitation for about 1.5 minutes, after which the strawberry pectin pieces are sprinkled into and mixed with the composition over the next 0.5 minutes. The resulting mass is slabbed, slit, and cut into 32 g bars, and then enrobed with yogurt coating to form a 40 g bar.

### Examples 7-11

Examples 7-11 illustrate nutritional bar embodiments of the present invention, all of which are also used in accordance with the various methods of the present invention. Each bar is prepared in a manner similar to that described above.

Examples 7-11: Nutrition Bars

| Ingredient | Example 7 | Example 8 | Example 9 | Example 10 | Example 11 |
|---|---|---|---|---|---|
| Rice Syrup 42 DE, High Maltose-WRSHM | 2.1251% | 2.1336% | 1.9947% | 3.5491% | 6.0308% |
| Brown Rice Syrup 42 DE, High Maltose/ | 1.6335% | 1.6400% | 1.6355% | 1.5589% | 1.3838% |
| BRSHMCL | | | | | |
| Energy Smart® Syrup (mixed fruit juice | 15.0606% | 15.1208% | 15.0787% | 14.3728% | 12.8508% |
| concentrates and natural grain dextrins) | | | | | |
| Glycerin | 1.0209% | 1.2136% | 1.2102% | 1.1536% | 1.0240% |
| Sugar Free Marshmallow Binder | 14.2938% | 14.3510% | 14.3132% | 13.6410% | 12.1088% |
| High Oleic Safflower Oil | 1.6335% | 1.6394% | 1.6355% | 1.5589% | — |
| Fructose | 0.0104% | 0.1042% | 0.1039% | 0.0991% | 0.0879% |
| Diced Almond Nut | 1.3282% | 1.3335% | — | | — |
| Diced Pecans | — | — | 0.7979% | | — |
| Peanut Flour | — | — | — | — | 3.5330% |
| Flavor | 1.3723 | 1.0048 | 1.4300 | 0.9239 | 0.4338 |
| Natural Peanut Flavored Powder | — | — | — | — | 0.3960% |
| Peanut Oil | — | — | — | — | 1.3838% |
| Lecithin- | — | — | — | — | 0.0315% |
| Maltodextrin (Fibersol 2) DE8-12 | 5.3127% | 5.3340% | 5.3192% | 3.0421% | 1.4177% |
| Vitamin-mineral premix | 0.5200% | 0.5414% | 0.4973% | 0.5197% | 0.4670% |
| Ascorbic Acid (70% active) | 0.0492% | 0.0494% | 0.0452% | 0.0469% | 0.0417% |
| Fructo Oligosaccharide (FOS) | 0.9799% | 0.9839% | 0.9811% | 0.9352% | 0.8301% |
| Cellulose Gel | 0.6535% | 0.6562% | 0.6543% | 0.6237% | 0.5536% |
| Dried Apple Pieces | 2.1251% | 2.1336% | — | — | — |
| Soy Protein Isolate Crisp (˜80% protein) | 7.2347% | 7.3269% | 7.2720% | 6.7524% | 4.4565% |
| Soy Guar Crisp (Example 2) | 23.6246% | 23.6310% | 23.6245% | 23.5850% | 23.6250% |
| Apple Cinnamon Pectin Fruit Pieces | 4.5158% | 4.5339% | — | — | — |
| (no sugar added) | | | | | |
| Butter Pecan Pectin | — | — | 7.1808% | — | — |
| (no sugar added) | | | | | |
| Chocolate Peanut Pectin Piece | — | — | — | — | 5.1757% |
| (no sugar added) | | | | | |
| Espresso Pectin Piece | — | — | — | 4.3096% | — |
| (no sugar added) | | | | | |
| Chocolate Chip Lactitol | — | — | — | 2.5351% | 2.2503% |
| Yogurt Coating (no sugar added) | 16.5058% | 16.2690% | 16.2255% | — | — |
| Chocolate Coating (no sugar added) | — | — | — | 20.7931% | 21.9180% |
| Total | 100.000% | 100.000% | 100.000% | 100.000% | 100.000% |

### Example 12

Example 12 illustrates layered nutrition bar embodiments of the present invention. Each composition is a strawberry layer bar with guar crisps and strawberry alginate filling. Each is also used in accordance with the various methods of the present invention.

Examples 12: Nutrition Bars

| Ingredient | Example 12 |
|---|---|
| Corn syrup 42 DE | 11.384% |
| Fructose, crystalline | 5.480% |
| White granulated Sugar | — |
| Marshmallow Crème No added sugar | 14.360% |
| Industrial Marshmallow crème | — |
| Marshmallow Flavor | 0.166% |
| Dicalcium phosphate dibasic | 1.000% |
| Guar Crisps (Example 1) | 23.000% |
| Soy Protein Isolate Crisps (80% protein) | — |
| Vitamin mineral premix | 0.410% |
| Ascorbic Acid (70% active) | 0.100% |
| Strawberry Paste-Sugar Free | 22.100% |
| Sodium alginate | 2.000% |
| Strawberry Filling A (8.7% sodium alginate) | — |
| Yogurt Coating No Sugar Added | 20.000% |
| Yogurt Coating with Sugar | — |
| Total | 100.000% |

Example 12 is prepared by similar methods. The Example 12 product, for example, is prepared by mixing together and heating to 120° F. the corn syrup, crystalline fructose, and marshmallow binder, and then once heated, the flavorant, marshmallow flavor, and dicalcium phosphate are mixed into the forming composition, followed by the addition of the crisps, vitamin premix, and ascorbic acid. The resulting mass if removed and formed into a slab. Strawberry paste and sodium alginate are then mixed together in a separate vessel and then spread or extruded on top of the slabbed crispy mixture. The resulting slabbed composition is then cut into 32 g bars and coated with the yogurt coating to form 40 g layered and coated nutritional bars.

### Example 13: Soy-Fiber Powder

The crisp matrix of example 5 is pulverized (i.e., reduced to powder or fine particulate form by any conventional or otherwise suitable technique) to form a soy-guar flour embodiment of the present invention, which may be formulated by conventional methods into most any solid nutrition product form, including nutrition bars and free flowing breakfast cereals, especially nutrition bars directed to meeting the medical nutritional needs of the individual afflicted with diabetes.

### EXPERIMENT I

A randomized, double-blinded, three-period crossover study is conducted to determine the postprandial glycemic response of adult type 2 diabetic subjects consuming an experimental IVF crispy bar compared to two commercially available crispy bars. Each subject received all three treatments in a randomized order and therefore served as his or her own control. In total, 62 subjects from 2 sites are enrolled for intent to treat analysis.

The IVF crispy bar, which is a specific embodiment of the present invention, is compared to the two commercial crispy bars—Pria® Nutritional Energy Snack Bar (NestleUSA, Glendale, Calif.) and Choice DM® Crispy Snack Bar (Mead Johnson Nutritionals). Each test bar provides 300 kcal. And each has a similar flavor and is cut into bite size pieces to look similar and to avoid any perception difference in the number of bars presented. The ingredient listings for the bars are as follows:

1. IVF Crispy Bar: soy protein nuggets (isolated soy protein, guar gum, rice flour, tapioca starch, salt, tricalcium phosphate), chocolate flavored coating (maltitol and/or lactitol, factionated palm kernel oil, polydextrose, cocoa, natural and artificial flavors, soy lecithin, monoglycerides, acesulfame K), fruit juice and grain dextrins, maltitol syrup, chocolate flavored chips (maltitol, chocolate liquor, cocoa butter, soy lecithin, vanilla), high maltose rice sryrup, corn maltodextrin, honey graham meal (enriched flour [wheat floour, niacin, reduced iron, thiamine mononitrate, riboflavin, folic acid], high fructose corn syrup, graham flour, vegetable shortening [partially hydroagenated soybean and/or canola oils], baking soda, salt, honey), less than 2% of the following: glycerine, high oleic safflower oil, high maltose brown rice syrup, fractionated palm kernel oil, natural and artificial flavors, fructooligosaccharides, water cellulose gel (celluose gum, guar gum), dl-alpha-tocopheryl acetate, ascorbic acid, egg white powder, fructose, titanium dioxide, lecithin, salt beta-carotene and chromium chloride.

2. Choice DM® Nutrition Bar (Mead Johnson Nutritionals): rice flour, hydrolyzed hydrogenated starch, lactitol, peanuts, fractionated palm kernel oil, high fructose corn syrup, fructose, soy protein isolate, corn starch, barley malt extract, partially defatted peanut flour, glycerin, figs, polydextrose, natural and artificial flavor, rice bran, calcium phosphate, maltitol syrup, cocoa, sorbitol, dextrose, salt, water, hydrogenated vegetable oil (cottonseed and rapeseed), alpha-tocopherol acetate, soy lecithin, monoglycerides, acesulfame K.

3. Pria® Nutritional Energy Snack Bar (NestleUSA, Glendale, Calif.): chocolatey coating (sugar, fractionated palm kernal oil, alkalized socoa, whey powder, nonfat milk powder, soy lecithin and vanilla), brown rice syrup, soy crisps (soy protein isolate, rice flour, malt and salt), rice crisps ( rice flour, malt and rice bran), soy protein isolate, graham cookies (sugar, wheat flour, graham flour, partially hydrogenated soybean and/or cottonseed oil, honey powder, sodium bicarbonate, salt, soy lecithin, natural flavor, caramel color, annatto color), rice flour, whole oats, natural flavors, glycerin, minerals (calcium carbonate, calcium phosphate, magnesium oxide, ferrous fumarate, zinc oxide, copper gluconate, manganese sulfate, chromium chloride, sodium selenite, potassium iodide and sodium molybdate), vitamins (vitamin C [ascorbic acid], vitamin E acetate, pantothenic acid [calcium pantothenate], vitamin B3 [niacinamide], vitamin B6 [pyroxidine hydrochloride], vitamin B2 [riboflavin], vitamin A [beta carotene], vitamin B1 [thiamine mononitrate], folic acid, biotin, vitamin K1 and vitamin B12.

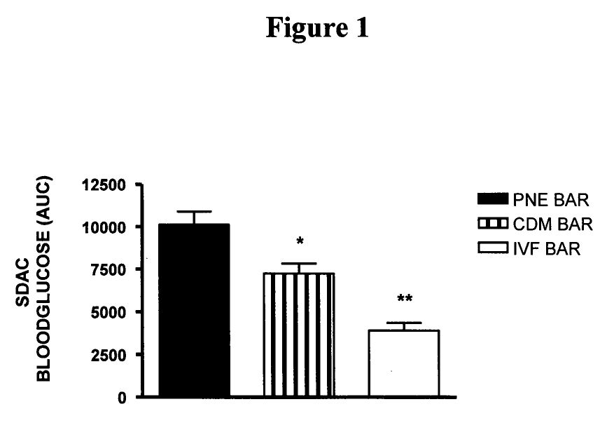

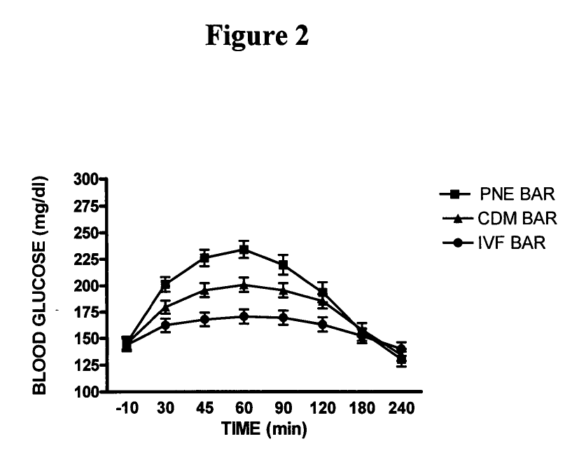

The postprandial glycemic response and area under the curve are significantly improved with the IVF Crispy Bar as compared to the two commericial bars, the results of which are summarized and illustrated in FIGS. 1 and 2.

### EXPERIMENT II

A study is conducted to assess the satiety effect of the IVF crispy bar of the present invention in adults with type 2 diabetes. The study is a two-treatment, double-blind, cross-over. The primary objective is to compare satiety after consumption of an IVF Crispy snack bar for lunch with that of a Choice DM® Nutrition Bar (Mead Johnson Nutritionals) by measuring subjective fullness in subjects with type 2 diabetes. In the study, 99 individuals are enrolled, of which 83 are protocol evaluable subjects.

At each treatment visit, overnight fasted subjects receive a standard breakfast consisting of Zone Perfect All Natural Nutrition Bars. Approximately 4 hours later, each consumes 300 kcal of study product for lunch and then rate various aspects of satiety (0 to 100 on a visual analog scale) every 30 minutes for 5 hours.

Study products used in this study include the IVF crispy bar of Experiment I and a commercially available crispy bar—Choice DM® Nutrition Bar (Mead Johnson Nutritionals). Each has a similar flavor and is cut into bite size pieces to look similar and to avoid any perception difference in the number of bars presented. The ingredient listing for the Choice DM® Nutrition Bar (Mead Johnson Nutritionals) is as follows:

Choice DM® Nutrition Bar (Mead Johnson Nutritionals): maltodextrin (includes resistant starch), rice syrup, cocoa processed with alkali, soy protein isolate, sugar, soy nuggets (soy protein isolate, rice flour, mali, salt), sorbitol, fructose, honey, palm kernel oil, calcium and sodium caseinate, canola oil, whey protein concentrate, lactose, high fructose corn syrup, milk minerals, malt syrup, chicory oligofructose, almond butter, natural and artificial flavor, dextrose, soy lecithin, and less than 1% vitamin E acetate, ascorbic acid, magnesium oxide, inositol, copper gluconate, ferric orthophosphate, biotin, beta-carotene, niacinamide, zinc oxide, vitamin K1, calcium pantothenate, vitamin B6 hydrochloride, riboflavin, vitamin A palmitate, manganese sulfate, thiamin mononitrate, vitamin D3, potassium iodide, chromic chloride, folic acid, sodium molybdate, sodium selenite, vitamin B12.

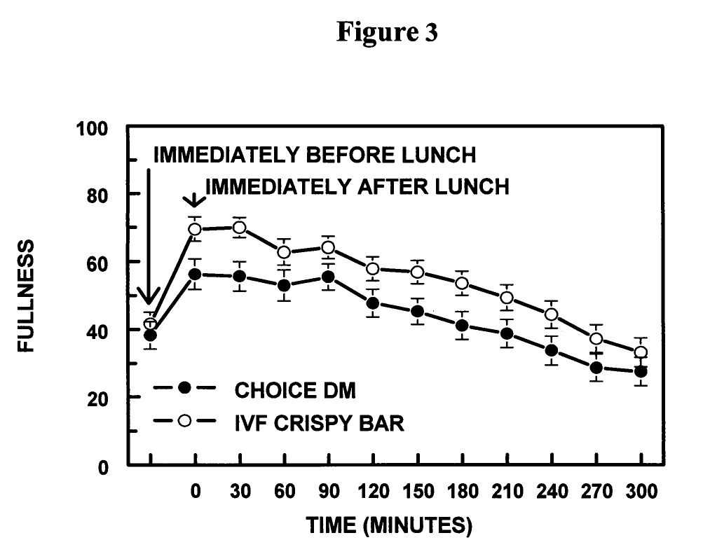

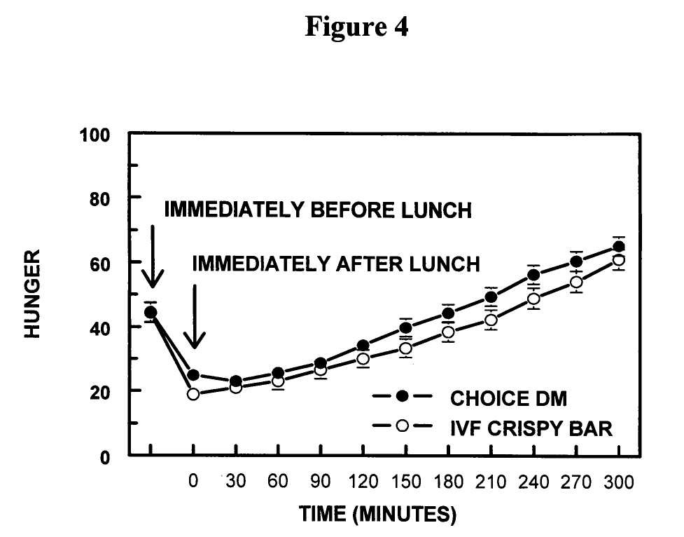

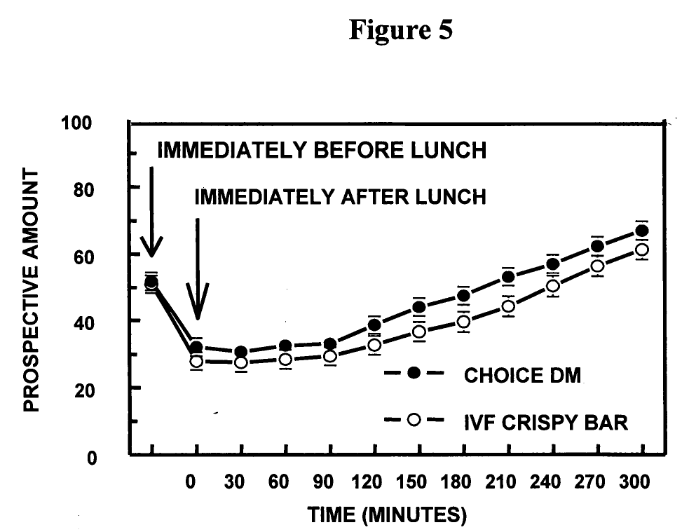

Study results are summarized and illustrated in FIGS. 3-5. From the data, a significant difference is detected between Choice DM® Nutrition Bar (Mead Johnson Nutritionals) and the IVF Crispy Bar for the fullness area under the curve (AUC) between 120 and 240 (mean±SEM; 4992.69±450.49 vs. 6345.68±395.27, respectively; p<0.001) and prospective consumption (5776.45±301.90 vs. 4863.61±340.88; p<0.001). Likewise, differences are also observed at the individual time points.

What is claimed is:

1. A nutritional composition having a solid crisp matrix, said matrix comprising:
(A) from about 10% to about 50% by weight of a soluble viscous fiber, and
(B) from about 10% to about 89% by weight of a carbohydrate in addition to the soluble viscous fiber, and
(C) from about 1% to about 49% by weight of protein;
wherein the solid crisp matrix has a bulk density of less than 0.4 g/cm³.

2. The nutritional composition of claim 1 wherein the soluble viscous fiber comprises guar gum.

3. The nutritional composition of claim 1 wherein the solid crisp matrix comprises from about 10% to about 50% by weight of guar gum.

4. The nutritional composition of claim 3 wherein the carbohydrate comprises rice starch.

5. The nutritional composition of claim 3 wherein the solid crisp matrix comprises from about 1% to about 49% by weight of soy protein.

6. The nutritional composition of claim 3 wherein the solid crisp matrix has a bulk density of from about 0.22 g/cm³ to about 0.28 g/cm³.

7. The nutritional composition of claim 3 wherein the composition further comprises, in addition to the solid crisp matrix, a solid crisp material selected from the group consisting of corn crisps, rice crisps, soy crisps, and combinations thereof, wherein the solid crisp material contains less than 9% by weight of a soluble viscous fiber and represents from about 5% to about 90% by weight of the combination of the solid crisp matrix and the solid crisp material.

8. The nutritional composition of claim 1 wherein the composition is in the form of free flowing crisp particulates.

9. The nutritional composition of claim 1 wherein the composition is reduced to powder form.

10. The nutritional composition of claim 3 wherein the composition is in bar form.

11. The nutritional composition of claim 1 wherein the composition further comprises from about 0.05% to about 3% by weight of an acidulant.

12. The nutritional composition of claim 11 wherein the acidulant is selected from the group consisting of citric acid, phosphoric acid, malic acid, fumaric acid, adipic acid, gluconic acid, tartaric acid, ascorbic acid, acetic acid, phosphoric acid, salts thereof, and combinations thereof

13. The nutritional composition of claim 1 wherein the composition further comprises from about 0.1% to about 10% by weight of a sour flavorant.

14. The nutritional composition of claim 1 wherein the composition further comprises from about 0.05% to about 3% by weight of an acidulant and from about 0.1% to about 10% by weight of a sour flavorant.

15. The nutritional composition of claim 1 wherein composition further comprises one or more gelled inclusions, said gelled inclusions comprising water and from about 0.5% to about 9% by weight of a gellant.

16. The nutritional composition of claim 15 wherein the gelled inclusions are individual particulates dispersed throughout the solid crisp matrix.

17. The nutritional composition of claim 15 wherein the gelled inclusions further comprise an acidulant selected from the group consisting of citric acid, phosphoric acid, malic acid, fumaric acid, adipic acid, gluconic acid, tartaric acid, ascorbic acid, acetic acid, phosphoric acid, salts thereof, and combinations thereof

18. The nutritional composition of claim 17 wherein gelled inclusions represent from about 1% to about 15% by weight of the nutritional composition, and wherein the gelled inclusions comprise from about 0.05% to about 3% of an acidulant and from about 0.05% to about 10% of a sour flavorant, all by weight of the nutritional composition.

19. The nutritional composition of claim 23 wherein the gellant is pectin.

20. A nutrition bar comprising a solid crisp matrix in combination with one or more gelled inclusions, the solid crisp matrix containing;
(A) from about 10% to about 50% of guar, and
(B) from about 10% to about 89% of a carbohydrate in addition to guar, and
(C) from about 1% to about 49% of soy protein,
and the gelled inclusions containing;
(D) water,
(E) from about 0.5% to about 9% of pectin by weight of the inclusions,
(F) from about 0.1% to about 10% of a sour flavorant by weight of the nutrition bar,
(G) from about 0.05% to about 3% of an acidulant by weight of the nutrition bar,
wherein the solid crisp matrix has a bulk density of from about 0.10 g/cm³ to about 0.30 g/cm³ and the gelled inclusions represent from about 1% to about 15% by weight of the nutrition bar.

21. A method of controlling blood glucose concentrations in a diabetic or other individual following a snack or meal, said method comprising the oral administration of the composition of claim 1.

22. A method of reducing appetite in a diabetic or other individual, said method comprising the oral administration of the composition of claim 1.
